(19) United States
(12) Patent Application Publication
(10) Pub. No.: US 2023/0030656 A1
(43) Pub. Date: Feb. 2, 2023

(54) IN SILICO GENOMIC VARIANT IDENTIFICATION

(71) Applicant: Foundation Medicine, Inc., Cambridge, MA (US)

(72) Inventor: Jason Darryl HUGHES, Providence, RI (US)

(73) Assignee: Foundation Medicine, Inc., Cambridge, MA (US)

(21) Appl. No.: 17/772,451

(22) PCT Filed: Oct. 29, 2019
(86) PCT No.: PCT/US2019/058646

§ 371 Date: Apr. 27, 2022

(51) Int. Cl. G16B 20/20; G16B 45/00; G06N 3/12

(52) U.S. Cl. CPC G16B 20/20; G16B 45/00; G06N 3/126

(57) ABSTRACT

The present disclosure is directed to in silico techniques for identifying of genomic variants, and more specifically to iterative graph-based techniques for identifying genomic variants. An exemplary electronic device comprises one or more processors; a memory: and one or more programs, wherein the one or more programs are stored in the memory and configured to be executed by the one or more processors, the one or more programs including instructions for constructing a first graph representation of at least a portion of a reference sequence; constructing a second graph representation based on sequence reads associated with an individual and the first graph representation, identifying one or more candidate variants based on the second graph representation; and repeating the process by the one or more processors in accordance with a determination that termination conditions are not met.

### FIELD

The present disclosure relates generally to computer techniques for identifying of genomic variants, and more specifically to iterative graph-based techniques for identifying genomic variants including but not limited to insertions, deletions, re-arrangements, or more complex variants of regions in a genome.

### BACKGROUND

Genomic testing shows significant promise towards developing better understanding of cancers and managing more effective treatment approaches. Genomic testing involves the sequencing of the genome of a patient's biological sample (which may contain cancer cells or cell-free nucleic acid products of cancer cells) and identifying any genomic variants in the sample vs. a reference genetic sequence. A genomic variant can include, for example, insertions, deletions, substitutions, rearrangements, or any combination thereof. Identifying and understanding these genomic variants as they are found in a specific patient's cancer may also help develop better treatments and help identify the best approaches (or exclude ineffective approaches) for treating specific cancer variants using genomic information.

Generally, biological samples are processed in a laboratory with various possible techniques, with the end goal of extracting and isolating DNA contained therein. That isolated DNA is sequenced, resulting in an electronic description of the DNA from the patient sample. Often, that electronic description is in the form of several thousand “reads.” A single read generally comprises a relatively short 50-150 bases) subsequence of the patient's DNA. In contrast, the entire human genome is approximately 3 billion bases long, and sub-regions of interest for the purposes of this application can be several tens of thousands bases long.

Currently, different tools (e.g., software programs) are needed for detecting different types of genomic variants in the acquired. sequence. For example, a software program may be specifically designed to identify only indels (i.e., insertion or deletion of bases in the genome) but is incapable of identifying other types of genomic variants such as substitutions and rearrangements. In addition to such inefficiencies, existing tools are often unable to identify complex and/or moderately sized variants, such as repetition of an entire region, deletion of an entire region, and re-arrangement of entire regions. For example, existing tools often cannot differentiate a large indel or substitution from a rearrangement.

Thus, there is a need for a system that can provide improved efficiency and accuracy over the existing tools. For example, there is a need for a system that can simultaneously detect multiple types of genomic variants, including insertions, deletions, substitutions, and rearrangements, in a single process. Further, there is a need for a system that can detect genomic variants of any size, including complex events and reduce missed and/or duplicated variant calls.

### BRIEF SUMMARY

An exemplary computer-enabled method for identifying a set of genomic variants in an individual comprises: (a) receiving a plurality of sequence reads associated with the individual; (b) identifying, based on a locus of interest on a reference sequence, a subset of the plurality of sequence reads associated with the individual; (c) constructing a first graph representation of at least a portion of the reference sequence, wherein the first graph representation comprises a plurality of reference nodes and wherein each reference node of the plurality of reference nodes has a same node length; (d) constructing a second graph representation based on the subset of the plurality of sequence reads associated with the individual and the first graph representation; (e) identifying one or more candidate variants based on the second graph representation; (f) adding the identified one or more candidate variants to the set of genomic variants; (g) determining whether one or more of a plurality of termination conditions is met, in accordance with a determination that none of the plurality of termination conditions is met: incrementing the node length by a predefined value; and repeating steps (c)-(g); in accordance with a determination one or more of the plurality of termination conditions are met: foregoing repeating steps (c)-(g).

In some embodiments, each of the first and the second graph representations is a De Bruijn graph.

In some embodiments, the plurality of sequence reads associated with an individual are from a sample acquired from the individual.

In some embodiments, the individual has a cancer chosen from a bladder cancer, a brain cancer, a breast cancer, a colon cancer, a hemangioblastoma, a liver cancer, a lung cancer, a melanoma, a neuroendocrine cancer, a pancreatic cancer, a retinoblastoma a stomach cancer, a thyroid cancer, a uterine or endometrial cancer, a Wilms' tumor, or an ovarian cancer.

in some embodiments, the method further comprises: before step (e), identifying a reference node having more than one instance in the first graph representation; and marking the reference node as an ambiguous node.

In some embodiments, the method further comprises: associating the reference node with a reconsideration list.

In some embodiments, identifying one or more candidate variants based on the second graph representation comprises: traversing a path diverging from a reference node in the second graph representation until a traversal termination condition of a plurality of traversal termination conditions is met.

In some embodiments, the traversal termination comprises a determination that the path includes an ambiguous node. In some embodiments, the method further comprises foregoing adding a candidate variant to a plurality of candidate variants based on the path.

In some embodiments, the traversal termination comprises a determination that the path includes a cycle. In some embodiments, the method further comprises foregoing adding a candidate variant to a plurality of candidate variants based on the path. In some embodiments, the method further comprises associating the cycle with a reconsideration list.

In some embodiments, the traversal termination comprises a determination that the path includes a dead end. In some embodiments, the method further comprises: adding a candidate variant to a plurality of candidate variants based on the path.

In some embodiments, the traversal termination comprises a determination that the path joins a reference node that is not an ambiguous node. In some embodiments, the method further comprises: adding a candidate variant to a plurality of candidate variants based on the path.

In some embodiments, identifying one or more candidate variants based on the second graph representation comprises: obtaining a plurality of candidate variants; and clustering the plurality of candidate variants.

In some embodiments, the method further comprises: updating the plurality of candidate variants by removing candidate variants belonging to a problematic cluster.

In some embodiments, the problematic cluster is identified based on one or more predefined rules.

In some embodiments, the method further comprises: updating the plurality of candidate variants by decomposing one or more candidate variants in the plurality of candidate variants.

In some embodiments, the plurality of termination conditions comprises a determination that node length exceeds a threshold.

In some embodiments, the plurality of termination conditions comprises a determination that no nodes or edges are associated with a reconsideration list.

In some embodiments, the method further comprises: classifying a genomic variant of the set of genomic variants to one of a plurality of categories.

In some embodiments, the plurality of categories comprises an insertion, a deletion, a substitution, a rearrangement, or any combination thereof.

In some embodiments, the method further comprises: identifying a variant of interest from the set of genomic variants.

In some embodiments, the method further comprises: directing a treatment based on the variant of interest.

In some embodiments, the method further comprises: providing an output indicative of a diagnosis based on the variant of interest.

In some embodiments, the method further comprises: providing one or more textual or graphical outputs based on the one or more candidate variants.

In some embodiments, identifying, based on the locus of interest on the reference sequence, the subset of the plurality of sequence reads associated with the individual comprises: conducting a preliminary alignment of the plurality of sequence reads with respect to the reference sequence.

An electronic device, comprises: one or more processors; a memory; and one or more programs, wherein the one or more programs are stored in the memory and configured to be executed by the one or more processors, the one or more programs including instructions for: (a) receiving a plurality of sequence reads associated with the individual; (b) identifying, based on a locus of interest on a reference sequence, a subset of the plurality of sequence reads associated with the individual; (c) constructing a first graph representation of at least a portion of the reference sequence, wherein the first graph representation comprises a plurality of reference nodes and wherein each reference node of the plurality of reference nodes has a same node length; (d) constructing a second graph representation based on the subset of the plurality of sequence reads associated with the individual and the first graph representation; (e) identifying one or more candidate variants based on the second. graph representation; (f) adding the identified one or more candidate variants to the set of genomic variants; (g) determining whether one or more of a plurality of termination conditions is met, in accordance with a determination that none of the plurality of termination conditions is met: incrementing the node length by a predefined value; and repeating steps (c)-(g); in accordance with a determination one or more of the plurality of termination conditions are met: foregoing repeating steps (c)-(g).

An exemplary non-transitory computer-readable storage medium stores one or more programs, the one or more programs comprising instructions, which when executed by one or more processors of an electronic device having a display, cause the electronic device to: (a) receive a plurality of sequence reads associated with the individual; (b) identify, based on a locus of interest on a reference sequence, a subset of the plurality of sequence reads associated with the individual; (c) construct a first graph representation of at least a portion of the reference sequence, wherein the first graph representation comprises a plurality of reference nodes and wherein each reference node of the plurality of reference nodes has a same node length; (d) construct a second graph representation based on the subset of the plurality of sequence reads associated with the individual and the first graph representation; (e) identify one or more candidate variants based on the second graph representation; (f) add the identified one or more candidate variants to the set of genomic variants; (g) determine whether one or more of a plurality of termination conditions is met, in accordance with a determination that none of the plurality of termination conditions is met: increment the node length by a predefined value; and repeat steps (c)-(g); in accordance with a determination one or more of the plurality of termination conditions are met: forego repeating steps (c)-(g).

### DESCRIPTION OF THE FIGURES

FIG. 1 depicts an exemplary graph-based process for identifying genomic variants, in accordance with some embodiments.

FIG. 2 depicts an exemplary sub-process of the exemplary graph-based process for identifying genomic variants, in accordance with some embodiments.

FIG. 3 depicts an exemplary process for performing a preliminary alignment of reads with respect to a reference sequence, in accordance with some embodiments.

FIG. 4A depicts an exemplary De Bruijn graph representation of a portion of a reference sequence, in accordance with some embodiments.

FIG. 4B depicts the exemplary De Bruijn graph representation updated with a sequence read associated with a patient, in accordance with some embodiments.

FIG. 4C depicts an exemplary De Bruijn graph representation of a portion of a reference sequence, in accordance with some embodiments.

FIG. 4D depicts the exemplary De Bruijn graph representation updated with a sequence read associated with a patient, in accordance with some embodiments.

FIG. 5 depicts different types of genomic variants represented in graph topology, in accordance with some embodiments.

FIG. 6 depicts exemplary data structures for tracking and identifying candidate genomic variants, in accordance with some embodiments.

FIG. 7 depicts a block diagram of an exemplary process for identifying genomic variants, in accordance with some embodiments.

FIG. 8 depicts an exemplary electronic system, in accordance with some embodiments.

### DETAILED DESCRIPTION

The present disclosure provides systems, devices, methods, apparatuses, and non-transitory computer-readable storage media for accurate and efficient discovery of genomic variants. Some embodiments of the present disclosure include a single graph-based computer-implemented algorithm for detecting multiple types of genomic variants including indels (i.e., insertion or deletion of bases in the genome), substitutions, rearrangements, or any combination thereof, at a given locus. In some embodiments, the algorithm includes constructing one or more graph representations (e.g., De Bruijn graphs) of patient-specific sequence reads. Also provided are systems, devices, and non-transitory computer-readable storage media for storing one or more programs for carrying out any one or more steps of the methods described herein.

In an exemplary embodiment, a system (e.g., one or more electronic devices) receives a plurality of sequence reads associated with the individual. The plurality of sequence reads can he derived from a sample associated with the individual (e.g., a patient). The system further receives a reference sequence, which can represent a person with or without a certain condition (e.g., a person who is cancer-free). The system then identifies, based on a locus of interest on a reference sequence, a subset of the plurality of sequence reads associated with the individual. In some embodiments, the system first uses a short read aligner to conduct a preliminary alignment of the plurality of reads with respect to the reference sequence, and identifies the subset of the plurality of sequence reads that is at or close to the locus of interest.

In a given iteration, the system constructs a first graph representation of at least a portion of the reference sequence. The first graph representation can be a De Bruijn graph comprising a plurality of reference nodes and each reference node of the plurality of reference nodes has a same node length. The system then constructs a second graph representation based on the subset of the plurality of sequence reads associated with the individual and the first graph representation. The second graph representation can be another De Bruijn graph, with the first graph representation as the backbone. Variants in the individual's sequence reads can cause one or more paths to diverge from the backbone in the second graph representation. Accordingly, the system identifies one or more candidate variants based on the second graph representation, and adds the identified one or more candidate variants to the set of genomic variants.

During the construction of the first graph representation and the second graph representation, the system can identify problematic nodes and edges in the graph representations and add them to a reconsideration list for further consideration at a higher k-mer level. At the end of the iteration, the system determines whether at least one of a plurality of termination conditions is met. An exemplary termination condition is a determination that the reconsideration list is empty; another exemplary termination condition is a determination that the value of k exceeds a predefined threshold. In accordance with a determination that at least one of the plurality of termination conditions is met, the system terminates. In accordance with a determination that none of the plurality of termination conditions is met, the system increments the node length (i.e., the value of k) by a predefined value; and starts a new iteration. At the higher k-mer level, items on the reconsideration list may be resolved and removed.

The present invention is advantageous over existing tools in multiple aspects. For example, the graph-based approach allows for simple and efficient computer-implemented representations and manipulation of sequence reads associated with the patient (e.g., via standard graph data structures and graph algorithms). Further, a single process for detecting multiple types of variants eliminates the need for multiple tools and duplicative analysis. Further still, the graph-based representation reduces false positives (e.g., by resolving a number of alignment problems) and enables detection of complex events. Embodiments of the invention can differentiate rearrangement events from “large” short variants and reduce missed and/or duplicated variant calls. Embodiments of the invention can further provide improved quantitation given that variants are detected in a coherent manner.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features. integers, steps, operations, elements, components, and/or groups thereof.

The term “if” is, optionally, construed to mean “when” or upon or “in response to determining” or “in response to detecting,” depending on the context. Similarly, the phrase “if it is determined” or “if [a stated condition or event] is detected” is, optionally, construed to mean “upon determining” or “response to determining” or “upon detecting [the stated condition or event]” or “in response to detecting [the stated condition or event],” depending, on the context.

Although the following description uses terms “first,” “second,” etc. to describe various elements, these elements should not be limited by the terms. These terms are only used to distinguish one element from another. For example, a first candidate variant could be termed a second candidate variant, and, similarly, a second candidate variant could be termed a first candidate variant, without departing from the scope of the various described embodiments. The first candidate variant and the second candidate variant are both candidate variants, but they are not the same candidate variant.

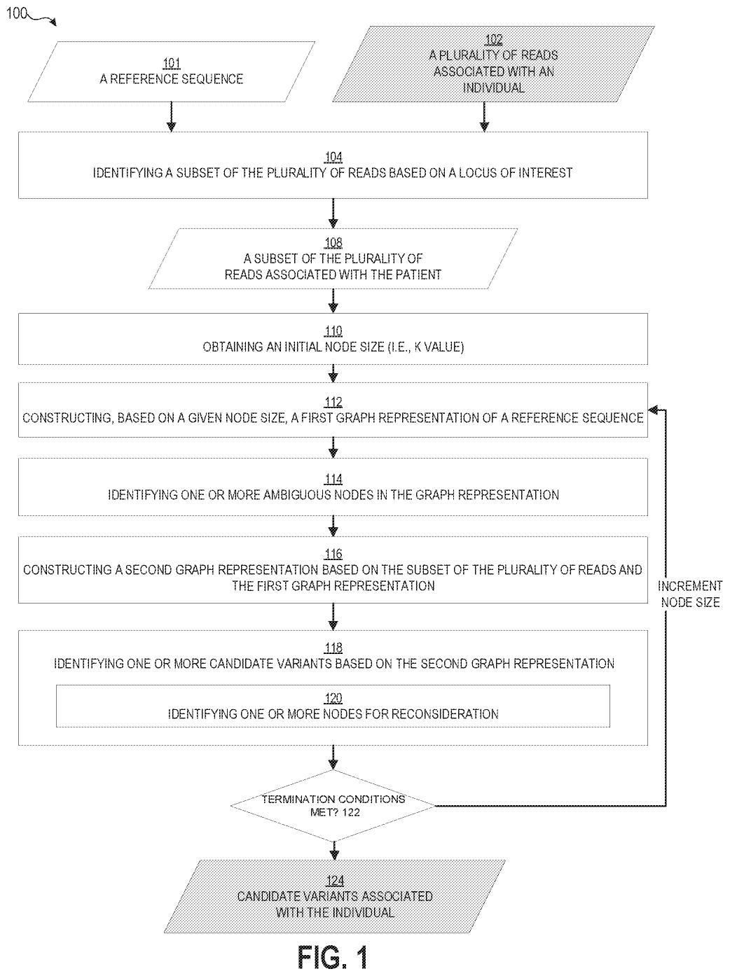

FIG. 1 illustrates an exemplary graph-based process 100 for identifying genomic variants, in accordance with some embodiments. Process 100 is performed, for example, using one or more electronic devices implementing a software program. In some examples, process 100 is performed using a client-server system, and the blocks of process 100 are divided up in any manner between the server and a client device, In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

With reference to FIG. 1, an exemplary system (e.g., one or more electronic devices) can perform the process 100 based on a reference sequence 101 and a plurality of sequence reads 102 associated with an individual (e.g., a patient). In process 100, the system can provide one or more candidate variants 124 associated with the individual as its outputs. The one or more candidate variants can be used for further assessment, for example, to classify or categorize the candidate variants (e.g., as insertion, deletion, substitution, or replacement). Subsequently, one or more variants of interest can be identified for guiding diagnosis and/or treatment of a condition (e.g., cancer), as further described herein.

In some embodiments, the reference sequence 101 represents a person with or without a certain condition (e.g., a person who is cancer-free). The reference sequence 101 can include a nucleic acid sequence assembled as a representative example of a species' set of genes. As the nucleic acid sequence can be assembled from the sequencing of DNA from a number of donors, the reference sequence 101 does not accurately represent the set of genes of any single person. Instead, the reference sequence 101 provides a haploid mosaic of different DNA sequences from multiple donors. In some embodiments, the reference sequence 101 is obtained from one or more public or private databases (e.g., the Human Genome Project). In some embodiments, the reference sequence 101 can be from a healthy tissue of an individual and the “sample” is derived from diseased (e.g., cancerous) tissue from the same individual.

In some embodiments, the plurality of sequence reads associated with an individual 102 is derived from a sample associated with the individual (e.g., a patient). The sample can be acquired from the individual (e.g., via tumor biopsy, via blood draw, via bone marrow aspirate, or via some other process). The sample can include a blood sample, a plasma sample, a tissue sample, or any other types of sample that may have nucleic acid sequences.

Sequencing techniques can be performed on the sample to obtain the plurality of sequence reads 102. Sequencing can be performed using any known sequencing method, such as single-molecule real-time sequencing, ion semiconductor sequencing, pyrosequencing, massively parallel signature sequencing, or sequencing-by-synthesis chemistry. An exemplary method of sequencing-by-synthesis chemistry is performed using an Illumina HiSeq 2500® sequencer or an Illumina HiSeq 4000® sequencer. In some embodiments, sequencing is performed using an Illumina HiSeq1000® sequencer, and Illumina HiSeqX® sequencer, Roche 454® sequencer, or Life Technologies Ion Proton® sequencing systems. Other methods of sequencing are known in the art. A read or sequence read generally refer to a series of nucleotide assignments (e.g., by base calling) made during a sequencing process. Such sequences may be derived from signal sequences (e.g., during primary analysis). Sequence reads can, but need not, describe contiguous portions of a polynucleotide. In some implementations, a sequence read can describe one end portion 50-150 bases) of a polynucleotide. In some implementations, a sequence read can describe both ends (e.g., 50-150 each) of a polynucleotide.

At block 104, the system identifies a subset of the plurality of reads 108 based on a locus of interest. The locus of interest refers to one or more locations on the reference sequence 101. In some embodiments, the locus of interest is selected (e.g., by a human user, automatically by a computer) from a plurality of pre-identified regions or sections of interest on the reference sequence. For example, the locus of interest can be a region or section previously known to be relevant to oncology; i.e., providing information about the presence or absence of a clinically relevant tumor, providing information about the expected efficacy of a particular therapy, etc. In some embodiments, the locus of interest can correspond to one or more exons of a gene. In some embodiments, the length of the locus of interest is between 100 bases to 10,000 bases (e.g., 100, 200, 300, . . . , 980, 990, 10000 bases). In some embodiments, the length of the locus of interest is between 100 bases to 5,000 bases.

In some embodiments, the system first uses a short read aligner to conduct a preliminary alignment of the plurality of reads 102 with respect to the reference sequence 101. In some embodiments, the short read aligner is the Burrows-Wheeler Aligner (“RWA”). It should be appreciated that the short read aligner can be selected from any existing sequence alignment software that can perform a preliminary alignment (i.e., mapping) of the plurality of reads onto the reference sequence.

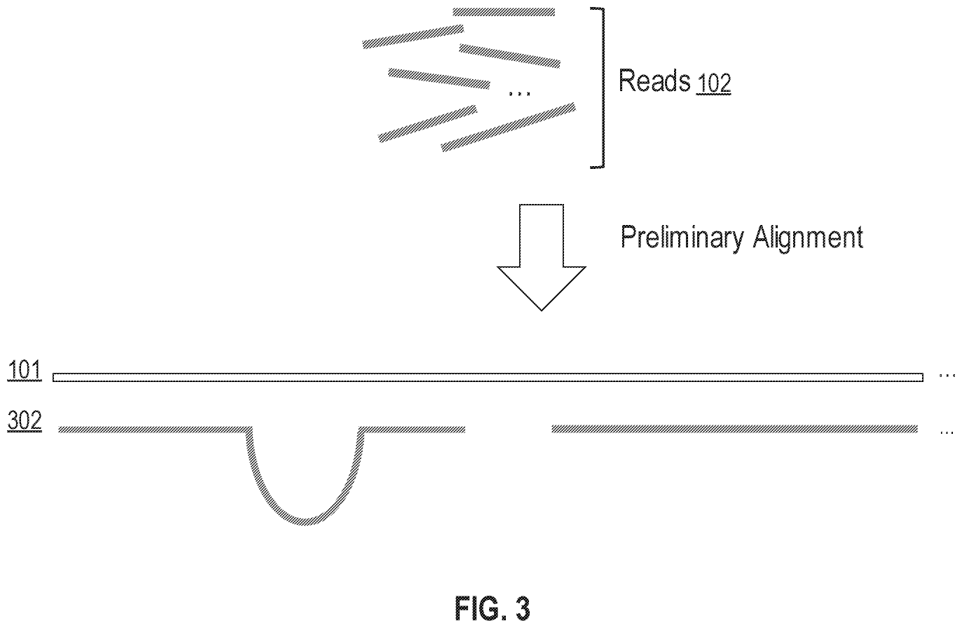

The output of the preliminary alignment step is a preliminary alignment of the plurality of reads 102 against the reference sequence 101, as shown in FIG. 3. At least some of the reads 102 may correspond to unique positions in the reference sequence 101. Moreover, at least some reads 102 may uniquely overlap with other reads 102, thus allowing the overlapping reads to be merged into a longer sequence 302, in some embodiments.

At the conclusion of the tough alignment, there is a relatively small number of relatively long sequences (compared to the number and length of the initial plurality of unaligned reads), such as the sequence 302. These long sequences may have areas of alignment with the reference sequence 101, as shown in FIG. 3 by parallel portions of the reference sequence 101 and the long sequence 302. Further, these sequences may also have areas of disagreement, as shown in FIG. 3 by a gap between the reference sequence 101 and the longer sequence 302 (signaling a potential deletion) and a curve on the longer sequence 302 representing a portion longer than on a corresponding portion of 101 (signaling a potential insertion). Areas of disagreement may correspond to variants in the patient sample vs. the reference sample, and can be further understood by the techniques described below.

After the preliminary alignment of the plurality of reads 102 is performed, the system selects, from the plurality of reads, a subset of reads 108 that are associated with the locus of interest. In some embodiments, the system selects reads that are at the locus of interest, in proximity to the locus of interest, or a combination thereof.

From blocks 110 to 122, the system identifies, from the subset 108 of the plurality of reads, one or snore genomic variants using an iterative graph-based technique.

At block 110, the system obtains an initial node length for constructing a graph representation for the first iteration. In some embodiments, the initial node length is obtained based on a user input. In some embodiments, the initial node length is automatically determined by the system. In some embodiments, the initial node length is between 5 to 20 bases (5, 6, . . . 19, 20). In some embodiments, the initial node length is 15 bases.

At block 112, the system constructs, based on the ode length, a first graph representation of the reference sequence for a given iteration. In some embodiments, the system constructs the first graphic representation corresponding only to a portion of the reference sequence associated with (e.g., at and/or close to) the locus of interest.

In some embodiments, the first graph representation is a De Bruijn graph. A De Bruijn graph is a directed graph comprising a plurality nodes and a plurality of directed edges, where each node is a k-mer (i.e., having a node length of k). Details of applying De Bruijn graphs in genome assembly can be found, for example, in “Assembly Algorithms for Next-Generation Sequencing Data” by Miller, Koren, and Sutton, the content of which is incorporated by reference in its entirety.

The first graph representation of the reference sequence forms the “backbone” of the De Bruijn graph. In some embodiments, the nodes belonging with the reference sequence (“reference nodes”) are each associated with an indicator (e.g., a label) that the respective node is part of the backbone. The graph representation of the reference sequence is constructed as a string of nodes, with neighboring nodes connected by a forward edge.

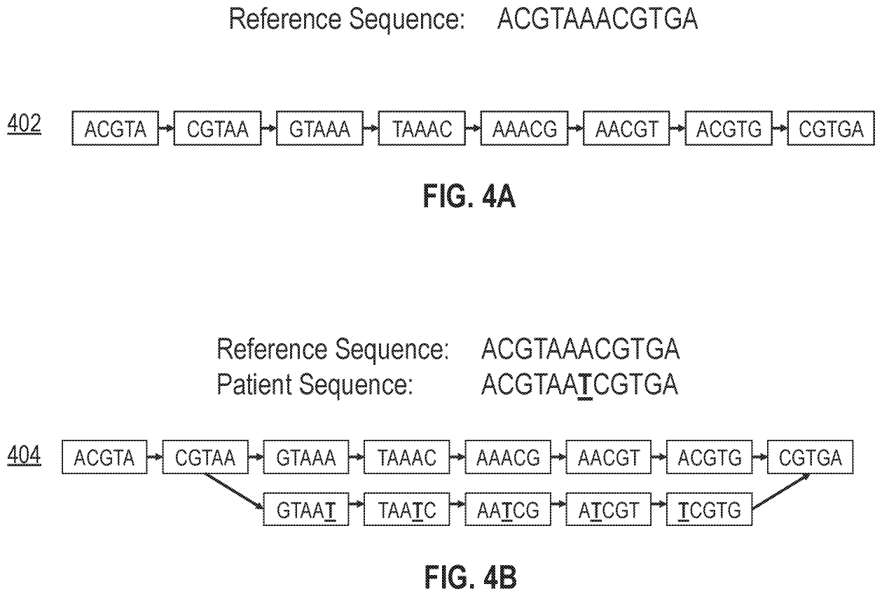

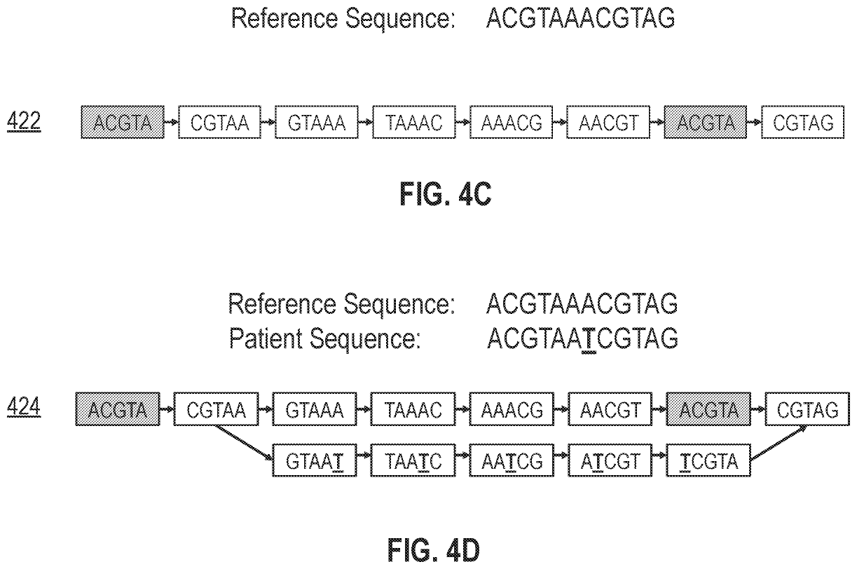

FIG. 4A illustrates an exemplary De Bruijn graph representation of a portion of a reference sequence, in accordance with some embodiments. As depicted, the reference sequence portion “ACGTAAACGTGA” is represented by a plurality of nodes (“ACGTA,” “CGTAA,” “GTAAA,”. . . . “CGTGA”) connected by a plurality of directed edges. Each node is of a node length of 5 bases and can be referred to as a “5-mer.” Each 5-mer is a part of the reference sequence portion, with neighboring 5-mers being offset by one base. The graph representation of the reference sequence (e.g., graph 402 in FIG. 4A) forms the backbone of the De Bruijn graph. Accordingly, each reference node in the graph 402 can be associated with a label indicating that it is part of the backbone.

in some embodiments, the graph representation is implemented using a graph data structure comprising one or more nodes and one or more edges. Each node or edge can be associated with one or more values. Further, each edge can be associated. with a direction.

Turning back to FIG. 1, at block 114, the system identifies one or more ambiguous nodes in the backbone. The system can identify an ambiguous node based on a predefined criteria. An exemplary criterion is a determination that a given node has multiple instances in the backbone. For example, with reference to FIG. 4C, for an exemplary reference sequence portion “ACGTAAACGTAG,” the De Bruijn graph includes 2 instances of node “ACGTA.” Accordingly, the node “ACGTA” (and/or a section around the node) is associated with an indicator that the node is an ambiguous reference node. As discussed below with reference to block 118, when a second graph representation is traversed to identify candidate variants, the system can stop traversing a path if it encounters a node corresponding to an ambiguous node.

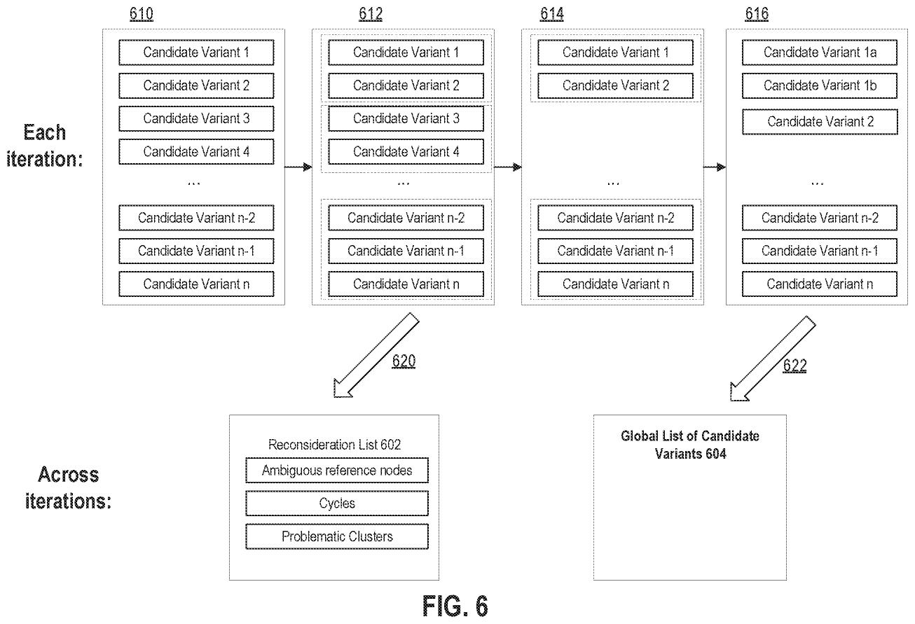

In some embodiments, the identified ambiguous reference nodes are added to a reconsideration list. As discussed below, the system maintains a reconsideration list (e.g., reconsideration list 602 in FIG. 6) across iterations to track problematic nodes and/or edges that need to be resolved in a future iteration, which include ambiguous reference nodes identified in a given iteration. For example, the ambiguous reference node “ACGTA” can be added to the reconsideration list. The ambiguous reference node can be represented in the reconsideration list by its position(s) in the reference sequence, its length, its sequence representation, its graphic representation, or a combination thereof.

At block 114, the system constructs a second graph representation based on the subset of the plurality of reads 108 and the first graph representation. The second graph representation can include an instance (e.g., a copy, a reference) of the first graph representation as the backbone. In some embodiments, the nodes corresponding to the reference nodes in the second graph representation are labelled as reference nodes. In some embodiments, the nodes corresponding to the ambiguous nodes are labelled as ambiguous nodes.

Further, a read can be represented by a plurality of k-mers, and the k-mers can be added to the instance of the first graph representation according to known assembly techniques based on De Bruijn graphs. In some embodiments, each node added to the backbone in block 114 is associated with indicators identifying the read(s) it comes from, its location in the read(s), and/or the path(s) it is a part of. In some embodiments, the system adds a read from the subset 108 to the second graph representation only if there are more than a predefined number of instances of the read (e.g., only if there are at least 2 copies of the read) in the subset 108.

FIG. 4B illustrates an exemplary second De Bruijn graph representation 404, in which an instance of the graph 402 forms the backbone and a patient sequence read “ACGTAATCGTGA” is then added. As depicted, the patient sequence read includes a substitution variant. The substitution variant causes a path diverging from the backbone (“GTAAT”→“TAATC”→“AATCG”→“ATCGT”→“TCGTG”) to he added to the backbone. The path then rejoins the backbone at the last node “CGTGA.”

FIG. 4D illustrates another exemplary second De Bruijn graph representation 424, in which an instance of the graph 422 forms the backbone and a patient sequence read “ACGTAATCGTAG” is then added. As depicted, the patient sequence read includes a substitution variant. The substitution variant causes a path diverging from the backbone (“GTAAT”→“TAATC”→“AATCG”→“ATCGT”→“TCGTA”) to be added to the backbone. The path then rejoins the backbone at the last node “CGTAG.” If the patient sequence read is such that the path rejoins the backbone at an ambiguous node (e.g., “ACETA”), the path is marked as a problematic path and marked for reconsideration at higher k-mer (e.g., added to the reconsideration list). The path can be represented in the reconsideration list by the sequence read it belongs to, its position in the read sequence, its length, its sequence representation, its graph representation (e.g., nodes and edges), its location relative to the reference sequence, or a combination thereof.

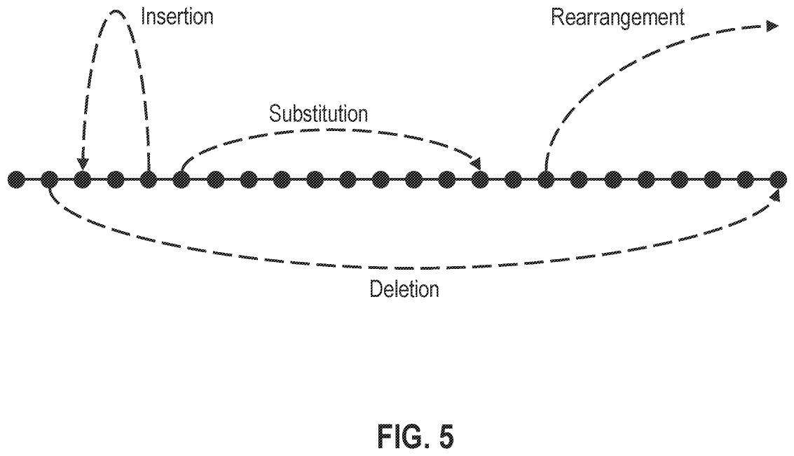

As illustrated in FIGS. 4A.-D, a substitution variant can result in a path diverging from the backbone at a first reference node and rejoining the backbone at a second reference node, where the second reference node is subsequent to the first reference node in the backbone. It should be appreciated that other types of variants can each cause a path to diverge from the backbone. FIG. 5 illustrates different types of genomic variants represented in graph topology, including insertion, deletion, and rearrangement. As depicted, an insertion can result in a path diverging from the backbone at a second reference node and rejoining the backbone at a first reference node, where the second reference node is subsequent to the first reference node in the backbone. A deletion can result in a path diverging from the backbone at a first reference node and rejoining the backbone at a second reference node, where the second reference node is subsequent to the first reference node in the backbone. A rearrangement can result in a path diverging from the backbone at a first reference node and not returning. Accordingly, each diverging path from the backbone in a De Bruijn graph can be indicative of a genomic variant.

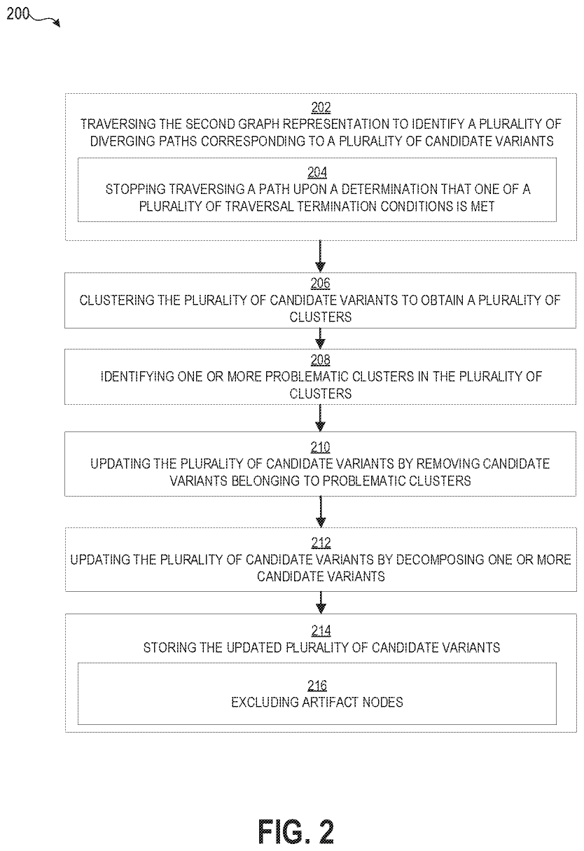

At block 118 the system identifies one or more candidate variants based on the second graph representation. Exemplary steps performed in block 118 are further described herein with reference to FIG. 2, in accordance with some embodiments. Process 200 is performed, for example, using one or more electronic devices implementing a software program. In some examples, process 200 is performed using a client-server system, and the blocks of process 200 are divided up in any manner between the server and a client device. In other examples, the blocks of process 200 are divided up between the server and multiple client devices. Thus, while portions of process 200 are described herein as being performed by particular devices of a client-server system, it will be appreciated that process 200 is not so limited. In other examples, process 200 is performed using only a client device or only multiple client devices. In process 200, some blocks are, optionally, combined, the order of some blocks is, optionally, changed, and some blocks are, optionally, omitted. In some examples, additional steps may be performed in combination with the process 200. Accordingly, the operations as illustrated (and described in greater detail below) are exemplary by nature and, as such, should not be viewed as limiting.

At block 202, the system traverses a second graph representation (e.g., obtained from block 116 in FIG. 1) to identify a plurality of diverging paths from the backbone. In some embodiments, the system identifies the backbone by using labels of the nodes in the second graph representation (e.g., identifying nodes labelled as reference nodes). In some embodiments, the system compares the second graph representation with the first graph representation (e.g., obtained from block 112 in FIG. 1) to identify the backbone. A diverging path refers to a path anchoring in a reference node in the backbone and leading away from the reference node. As illustrated in FIGS. 4A-4D, each diverging path can represent a candidate variant, such as an insertion, a deletion, a substitution, a rearrangement, or a combination thereof.

In some embodiments, the system traverses only diverging paths in the graph representation and ignores the path along the backbone. For example, with reference to FIG. 4B, the system traverses only the diverging path starting from “CGTAA” and ending at “CGTGA” and ignores the backbone portion between these two reference nodes.

At block 204, as the system traverses each diverging path, it determines whether one of a plurality of traversal termination conditions is met, as discussed

In some embodiments, the plurality of traversal termination conditions can include a determination that the path stops (e.g., includes a dead end). For example, if the system encounters a node that does not lead to another node in the path, the system stops traversing the path. The diverging path can be added to a list of candidate variants for further analysis. FIG. 6 depicts an exemplary list of candidate variants 610 onto which the diverging path can be added, as discussed further below.

In some embodiments, the plurality of traversal termination conditions can include a determination that an ambiguous reference node has been encountered on the path. Accordingly, the system stops traversing the path and does not add the path to the candidate variants. This way, candidate variants are only identified with respect to portions of the backbone that are not ambiguous.

In some embodiments, the plurality of traversal termination conditions can include a determination that a cycle has been encountered on the path. Cycle refers to repetitive sequences (short repeats longer than k-mer size) or longer cycles not in reference sequence. For example, if the system encounters a series of nodes on the path that it has previously traversed, the system stops traversing the path and does not add the path to the candidate variants. In some embodiments, the system adds the detected cycle to a reconsideration list reconsideration list 602 in FIG. 6). The cycle can be represented in the reconsideration list by the sequence read it belongs to, its position in the read sequence, its length, its sequence representation, its graph representation (e.g., nodes and edges), its location relative to the reference sequence, or a combination thereof.

in some embodiments, the plurality of traversal termination conditions can include a determination that the path has returned, to the backbone of the graph at a reference node that is not an ambiguous node. For example, if the system encounters a reference node in the backbone and the reference node is not labelled as an ambiguous reference node, the system stops traversing the path. The diverging path can be added to a list of candidate variants for further analysis,

After the system traverses all of the diverging paths in the second graph representation in block 202, the system obtains a plurality candidate variants. FIG. 6 illustrates an exemplary plurality of candidate variants 610 obtained after block 202. In some embodiments, each variant is represented by a graph-based representation and a sequence-based representation in the list 610. The graph-based representation includes a diverging path comprising nodes and directed edges. In some embodiments, the plurality of candidate variants 610 can be saved in a file format for storing genome sequences (e.g., Variant Call Format).

Blocks 206-212 include additional steps taken to further process the plurality of candidate variants. At block 206, the system clusters the plurality of candidate variants. Two candidate variants can be clustered together if one or more predefined criteria are met. In some embodiments, two candidate variants are clustered together if they share a non-reference node. In some embodiments, two candidate variants are clustered together if they are very close together in the reference sequence (e.g., if their distance does not exceed a predefined threshold). As an example, FIG. 6 depicts the plurality of candidate variants grouped in multiple clusters in 612.

At block 208, the system identifies one or more problematic clusters in the plurality of clusters. In some embodiments, a cluster is problematic if it contains a problematic candidate variant. A candidate variant is problematic if it contains ambiguous or cyclic nodes, spans a portion of the reference sequence having ambiguous nodes; includes short paths, is proximate (e.g., distance is smaller than a predefined threshold) to another path likely to merge at higher k-mer; or includes other nodes or patterns that are added to the reconsideration list.

At block 210, the system updates the plurality of candidate variants by removing candidate variants belonging to problematic clusters. In some embodiments, every candidate variant in a problematic cluster is marked for reconsideration (e.g., added to the reconsideration list) if any one path is found to be problematic. As an example, FIG. 6 depicts that a cluster of candidate variants (i.e., Candidate Variants 3 and 4) removed in the list 614. Further, the removed cluster of candidate variants can be added to the reconsideration list 602, as indicated by arrow 620. Each removed candidate variant can be represented in the reconsideration list by the sequence read it belongs to, its position in the read sequence, its length, its sequence representation, its graph representation (e.g., nodes and edges), its location relative to the reference sequence, or a combination thereof.

At block 212, the system updates the plurality candidate variants by decomposing one or more candidate variants. In some embodiments, the system decomposes complex variants (e.g., a variant in which a large sequence replaces another large sequence, a variant in which somatic and germline mutations are combined).

In some embodiments, the plurality of candidate variants are converted to sequence space. A multiple sequence alignment is generated for the reference and all alternate sequences. If the MSA has one or more sections (e.g., 5bp or more) of invariant sequence aligned, then variant decomposition is performed. First, the MSA is split at the invariant location. Separate variants are then generated for each slice of the MSA. Summary statistics (ref/alt kmer counts) are aggregated to the new variants. As an example, FIG. 6 depicts that a candidate variant (i.e., Candidate Variant 1) is decomposed into two candidate variants.

At block 214, the system stores the updated plurality of candidate variants. In some embodiments, the system maintains a global list of candidate variants (e.g., global list 604 in FIG. 6) across iterations and updates the global list based on the updated plurality of candidate variants after block 212. Each candidate variant can include a graph-based representation (i.e., nodes and edges) and a sequence-based representation, along with other associated information (e.g., chromosome position).

In some embodiments, before adding the updated plurality of candidate variants to the global list, the system determines whether a candidate variant from the updated plurality of candidate variants has been previously discovered (e.g., by checking the suffix of the nodes of the candidate variant). If the system determines that a candidate variant has been previously discovered, at a lower k-mer level and is already included in the global list, the system does not update the global list. In other words, the system always represents a candidate variant using the lower k-mer level at which the candidate variant is discovered.

In some embodiments, the system detects and excludes artifacts, which do not correspond to real genomic variants, from the candidate variants. An exemplary artifact is inversion, which can manifest as nodes connecting forward reverse-complement nodes. In some embodiments, if a path includes artifacts over a predefined threshold, the system can mask the path without removing the corresponding nodes and edges.

Turning back to FIG. 1, at block 122, the system determines whether at least one of a plurality of termination conditions are met. An exemplary termination condition is a determination that the reconsideration list is empty (i.e., all the items in the list have been resolved). Another exemplary termination condition is a determination that the value of k exceeds a predefined threshold. In some embodiments, the predefined threshold is between 90 and 100 (e.g., 90, 91, , 99, 100). In some embodiments, the predefined, threshold is obtained based on a user input.

If none of the termination conditions are met, blocks 112-122 are repeated in a new iteration. The value of k is incremented by a predefined amount (e.g., 8). In the new iteration, a new instance of the De Bruijn graph is constructed. Based on the new instance of the De Bruijn graph, additional candidate variants can be discovered and one or more items (e.g., ambiguous reference nodes, cycles, problematic variants) on the reconsideration list can be resolved and removed from the list.

If at least one termination condition is met, the process 100 terminates. The global list of candidate variants can be outputted via textual, graphical, and/or audio outputs. The global list of candidate variants can be further analyzed. to classify or categorize the candidate variants. For example, a candidate variant can be converted into a sequence, mapped to the genome, and aligned in multiple ways to characterize the junction (e.g., as an insertion, deletion, substitution, rearrangement). Further, variants of interest can he identified for use in diagnosis and/or treatment of cancer. For example, one genomic variant can be known to be associated with a certain type of tumor and/or respond to a certain treatment. Accordingly, once the genomic variant is identified, the patient can be diagnosed and/or prescribed a treatment accordingly.

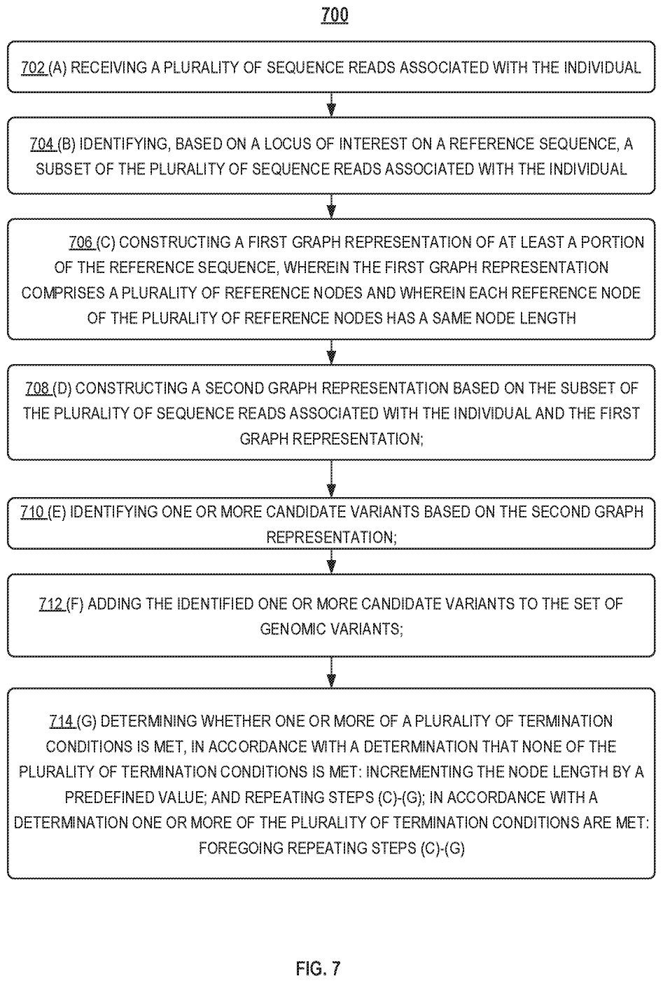

FIG. 7 illustrates an exemplary process 700 for identifying a set of genomic variants in an individual, in accordance with some embodiments. Process 700 is performed, for example, using one or more electronic devices implementing a software program. In some examples, process 700 is performed using a client-server system, and the blocks of process 700 are divided up in any manner between the server and a client device. In other examples, the blocks of process 700 are divided up between the server and multiple client devices. Thus, while portions of process 700 are described herein as being performed by particular devices of a client-server system, it will be appreciated that process 700 is not so limited. In other examples, process 700 is performed using only a client device or only multiple client devices. In process 700, some blocks are, optionally, combined, the order of some blocks is, optionally, changed, and some blocks are, optionally, omitted. In some examples, additional steps may be performed in combination with the process 700, Accordingly, the operations as illustrated (and described in greater detail below) are exemplary by nature and, as such, should not be viewed as limiting.

At block 702, an exemplary system (e.g., one or more electronic devices) receives a plurality of sequence reads associated with the individual. At block 704, the system identifies, based on a locus of interest on a reference sequence, a subset of the plurality of sequence reads associated with the individual. At block 706, the system constructs a first graph representation of at least a portion of the reference sequence, wherein the first graph representation comprises a plurality of reference nodes and wherein each reference node of the plurality of reference nodes has a same node length. At block 708, the system constructs a second graph representation based on the subset of the plurality of sequence reads associated with the individual and the first graph representation. At block 710, the system identifies one or more candidate variants based on the second graph representation. At block 712, the system adds the identified one or more candidate variants to the set of genomic variants. At block 714, the system determines whether one or more of a plurality of termination conditions is met, in accordance with a determination that none of the plurality of termination conditions is met: incrementing the node length by a predefined value; and repeating steps 706-714; in accordance with a determination one or more of the plurality of termination conditions are met: foregoing repeating steps 706-714.

In some embodiments, each of the first and the second graph representations is a De Bruijn graph.

In some embodiments, the plurality of sequence reads associated with an individual are from a sample acquired from the individual.

In some embodiments, the individual has a cancer chosen from a bladder cancer, a brain cancer, a breast cancer, a colon cancer, a hemangioblastoma, a liver cancer, a lung cancer, a melanoma, a neuroendocrine, cancer, a pancreatic cancer, a retinoblastoma, a stomach cancer, a thyroid cancer, a uterine or endometrial cancer, a Wilms' tumor, or an ovarian cancer.

In some embodiments, the method further comprises: before step 710, identifying a reference node having more than one instance in the first graph representation; and marking the reference node as an ambiguous node.

In some embodiments, the method further comprises: associating the reference node with a reconsideration list.

In some embodiments, identifying one or more candidate variants based on the second graph representation comprises: traversing a path diverging from a reference node in the second graph representation until a traversal termination condition of a plurality of traversal termination conditions is met.

In some embodiments, the traversal termination comprises a determination that the path includes an ambiguous node. In some embodiments, the method further comprises foregoing adding a candidate variant to a plurality of candidate variants based on the path.

In some embodiments, the traversal termination comprises a determination that the path includes a cycle. In some embodiments, the method further comprises foregoing adding a candidate variant o a plurality of candidate variants based on the path. In sone embodiments, the method further comprises associating the cycle with a reconsideration list.

In some embodiments, the traversal termination comprises a determination that the path includes a dead end. In some embodiments, the method further comprises: adding a candidate variant to a plurality of candidate variants based on the path.

In some embodiments, the traversal termination comprises a determination that the path joins a reference node that is not an ambiguous node. In some embodiments, the method further comprises: adding a candidate variant to a plurality of candidate variants based on the path.

In some embodiments, identifying e or more candidate variants based on the second graph representation comprises: obtaining a plurality of candidate variants; and clustering the plurality of candidate variants.

In some embodiments, the method further comprises: updating the plurality of candidate variants by removing candidate variants belonging to a problematic cluster.

In some embodiments, the problematic cluster is identified based. on one or more predefined rules.

In some embodiments, the method further comprises: updating the plurality of candidate variants by decomposing one or more candidate variants in the plurality of candidate variants.

In some embodiments, the plurality of termination conditions comprises a determination that node length exceeds a threshold.

In some embodiments, the plurality of termination conditions comprises a determination that no nodes or edges are associated with a reconsideration list.

In some embodiments, the method further comprises: classifying a genomic variant of the set of genomic variants to one of a plurality of categories.

In some embodiments, the plurality of categories comprises an insertion, a deletion, a substitution, a rearrangement, or any combination thereof.

In some embodiments, the method further comprises: identifying a variant of interest from the set of genomic variants.

In some embodiments, the method further comprises: directing a treatment based on the variant of interest.

In some embodiments, the method further comprises: providing an output indicative of a diagnosis based on the variant of interest.

In some embodiments, the method further comprises: providing one or more textual or graphical outputs based on the one or more candidate variants.

In sonic embodiments, identifying, based on the locus of interest on the reference sequence, the subset of the plurality of sequence reads associated with the individual comprises: conducting a preliminary alignment of the plurality of sequence reads with respect to the reference sequence.

In some embodiments, an electronic device comprises: one or more processors; a memory; and one or more programs, wherein the one or more programs are stored in the memory and configured to be executed by the one or more processors, the one or more programs including instructions for: (a) receiving a plurality of sequence reads associated with the individual; (b) identifying, based on a locus of interest on a reference sequence, a subset of the plurality of sequence reads associated with the individual; (c) constructing a first graph representation of at least a portion of the reference sequence, wherein the first graph representation comprises a plurality of reference nodes and wherein each reference node of the plurality of reference nodes has a same node length; (d) constructing a second graph representation based on the subset of the plurality of sequence reads associated with the individual and the first graph representation; (e) identifying one or more candidate variants based on the second graph representation; (f) adding the identified one or more candidate variants to the set of genomic variants; (g) determining whether one or more of a plurality of termination conditions is met, in accordance with a determination that none of the plurality of termination conditions is met: incrementing the node length by a predefined value; and repeating steps (c)-(g); in accordance with a determination one or more of the plurality of termination conditions are met: foregoing repeating steps (c)-(g).

In some embodiments, an exemplary non-transitory computer-readable storage medium stores one or more programs, the one or more programs comprising instructions, which when executed by one or more processors of an electronic device having a display, cause the electronic device to: (a) receive a plurality of sequence reads associated with the individual; (b) identify, based on a locus of interest on a reference sequence, a subset of the plurality of sequence reads associated with the individual; (c) construct a first graph representation of at least a portion of the reference sequence, wherein the first graph representation comprises a plurality of reference nodes and wherein each reference node of the plurality of reference nodes has a same node length; (d) construct a second graph representation based on the subset of the plurality of sequence reads associated with the individual and the first graph representation; (e) identify one or more candidate variants based on the second graph representation; (f) add the identified one or more candidate variants to the set of genomic variants; (g) determine whether one or more of a plurality of termination conditions is met, in accordance with a determination that none of the plurality of termination conditions is net; increment the node length by a predefined value; and repeat steps (c)-(g); in accordance with a determination one or more of the plurality of termination conditions are met: forego repeating steps (c)-(g).

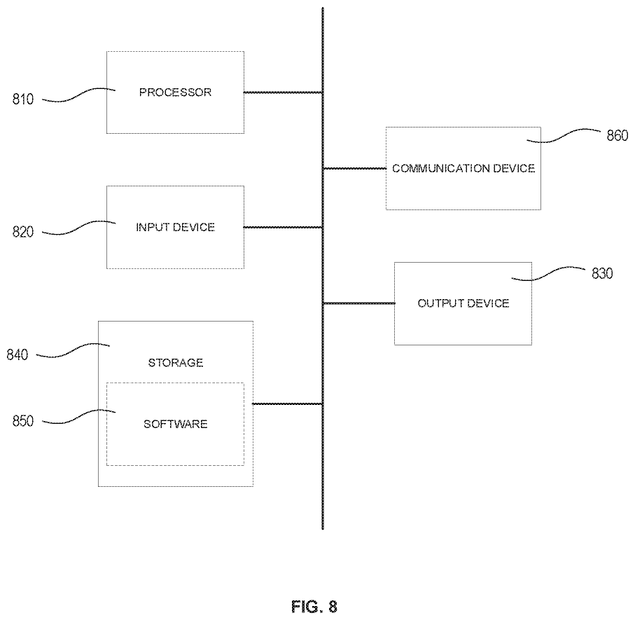

FIG. 8 illustrates an example of a computing device in accordance with one embodiment. Device 800 can be a host computer connected to a network. Device 800 can be a client computer or a server. As shown in FIG. 8, device 800 can be any suitable type of microprocessor-based device, such as a personal computer, workstation, server or handheld computing device (portable electronic device) such as a phone or tablet. The device can include, for example, one or more of processor 810, input device 820, output device 830, storage 840, and communication device 860. Input device 820 and output device 830 can generally correspond to those described above, and can either be connectable or integrated with the computer.

Input device 820 can be any suitable device that provides input, such as a touch screen, keyboard or keypad, mouse, or voice-recognition device. Output device 830 can be any suitable device that provides output, such as a touch screen, haptics device, or speaker.

Storage 840 can be any suitable device that provides storage, such as an electrical, magnetic or optical memory including a RAM, cache, hard drive, or removable storage disk. Communication device 860 can include any suitable device capable of transmitting and receiving signals over a network, such as a network interface chip or device. The components of the computer can be connected in any suitable manner, such as via a physical bus or wirelessly.

Software 850, which can be stored in storage 840 and executed by processor 810, can include, for example, the programming that embodies the functionality of the present disclosure (e.g., as embodied in the devices as described above).

Software 850 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions, in the context of this disclosure, a computer-readable storage medium can be any medium, such as storage 840, that can contain or store programming for use by or in connection with an instruction execution system, apparatus, or device.

Software 850 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 800 can implement any operating system suitable for operating on the network. Software 850 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

Exemplary methods, non-transitory computer-readable storage media, systems, and electronic devices are set out in the following items:

1. A computer-enabled method for identifying a set of genomic variants in an individual, the method comprising:
(a) receiving a plurality of sequence reads associated with the individual;
(b) identifying, based on a locus of interest on a reference sequence, a subset of the plurality of sequence reads associated with the individual;
(c) constructing a first graph representation of at least a portion of the reference sequence, wherein the first graph representation comprises a plurality of reference nodes and wherein each reference node of the plurality of reference nodes has a same node length;
(d) constructing a second graph representation based on the subset of the plurality of sequence reads associated with the individual and the first graph representation;
(e) identifying one or mare candidate variants based on the second graph representation;
(f) adding the identified one or more candidate variants to the set of genomic 7a s;
(g) determining whether one or more of a plurality of termination conditions is met, in accordance with a determination that none of the plurality of termination conditions is met:
   incrementing the node length by a predefined value; and
   repeating steps (c)-(g);

in accordance with a determination one or more of the plurality of termination conditions are met:

foregoing repeating steps (c)-(g).

2. The method of item 1, wherein each of the first and the second graph representations is a De Bruijn graph.

3. The method of any of items 1-2, wherein the plurality of sequence reads associated with an individual are from a sample acquired from the individual.

4. The method of any of items 1-3 wherein the individual has a cancer chosen from a bladder cancer, a brain cancer, a breast cancer, a colon cancer, a hemangioblastoma, a liver cancer, a lung cancer, a melanoma, a neuroendocrine cancer, a pancreatic cancer, a retinoblastoma, a stomach cancer, a thyroid cancer, a uterine or endometrial cancer, a Wilms' tumor, or an ovarian cancer.

5. The method of any of items 1-4, further comprising: before step (e), identifying a reference node h g more than one instance in the first graph representation; and
marking the reference node as an ambiguous node.

6. The method of item 5, further comprising: associating the reference node with a reconsideration list.

7. The method of any of items 1-7, wherein identifying one or more candidate variants based on the second graph representation comprises: traversing a path diverging from a reference node in the second graph representation until a traversal termination condition of a plurality of traversal termination conditions is met.

8. The method of item 8, wherein the traversal termination comprises a determination that the path includes an ambiguous node.

9. The method of item 9, further comprising: foregoing adding a candidate variant to a plurality of candidate variants based on the path.

10. The method of item 8, wherein the traversal termination comprises a determination that the path includes a cycle.

11. The method of item 11, further comprising: foregoing adding a candidate variant to a plurality of candidate variants based on the path.

12. The method of any of items 10-11, further comprising: associating the cycle with a reconsideration list.

13. The method of item 8, wherein the traversal termination comprises a determination that the path includes a dead end.

14. The method of item 13, further comprising: adding a candidate variant o a plurality of candidate variants based on the path.

15. The method of item 8, wherein the traversal termination comprises a determination that the path joins a reference node that is not an ambiguous node.

16. The method of item 15, further comprising: adding a candidate variant o a plurality of candidate variants based on the path.

17, The method of any of items 1-16, wherein identifying e or more candidate variants based on the second graph representation comprises:
obtaining a plurality of candidate variants; and
clustering the plurality of candidate variants.

18. The method of item 17, further comprising: updating the plurality of candidate variants by removing candidate variants belonging to a problematic cluster.

19. The method of item 18, wherein the problematic cluster is identified based on one or more predefined rules.

20, he method of item 18, further comprising: updating the plurality of candidate variants by decomposing one or more candidate variants in the plurality of candidate variants.

21. The method of any of items 1-20, wherein the plurality of termination conditions comprises a determination that node length exceeds a threshold.

22. The method of any of items 1-20, wherein the plurality of termination conditions comprises a determination that no nodes or edges are associated with a reconsideration list.

23. The method of any of items 1-22, further comprising: classifying a genomic variant of the set of genomic variants to one of a plurality of categories.

24, The method of any of items 23, wherein the plurality of categories comprises an insertion, a deletion, a substitution, a rearrangement, or any combination thereof.

25. The method of any of items 1-23, further comprising: identifying a variant of interest from the set of genomic variants.

26, he method of item 25, further comprising: directing a treatment based on the variant of interest.

27. The method of item 25, further comprising: providing an output indicative of a diagnosis based on the variant of interest.

28. The method of any of items 1-27, further comprising: providing one or more textual or graphical outputs based on the one or more candidate variants.

29. The method of any of items 1-28, wherein identifying, based on the locus of interest on the reference sequence, the subset of the plurality of sequence reads associated with the individual comprises; conducting a preliminary alignment of the plurality of sequence reads with respect to the reference sequence.

30. An electronic device, comprising;
one or n e processors;
a memory; and
one or more programs, wherein the one or more programs are stored. in the memory and configured to be executed by the one or more processors, the one or more programs including instructions for:
(a) receiving a plurality of sequence, reads associated with the individual
(b) identifying, based on a locus of interest on a reference sequence, a subset of the plurality of sequence reads associated. with the individual;
(c) constructing a first graph representation of at least a portion of the reference sequence, wherein the first graph representation comprises a plurality of reference nodes and wherein each reference node of the plurality of reference nodes has a same node length;
(d) constructing a second graph representation based on the subset of the plurality of sequence reads associated with the individual and the first graph representation;
(e) identifying one or more candidate variants based on the second graph representation;
(f) adding the identified one or more candidate variants to the set of genomic variants;
(g) determining whether one or more of a plurality of termination conditions is met, in accordance with a determination that none of the plurality of termination conditions is met:
   incrementing the node length by a predefined value; and
   repeating steps (c)-(g);
   in accordance with a determination one or more of the plurality of termination conditions are met:
   foregoing repeating steps (c)-(g) .

31. A non-transitory computer-readable storage medium storing one or more programs, the one or more programs comprising instructions, which when executed by one or more processors of an electronic device having a display, cause the electronic device to:
(a) receive a plurality of sequence reads associated with the individual;
(b) identify, based on a locus of interest on a reference sequence, a subset of the plurality of sequence reads associated with the individual;
(c) construct a first graph representation of at least a portion of the reference sequence, wherein the first graph representation comprises a plurality of reference nodes and wherein each reference node of the plurality of reference nodes has a same node length;
(d) construct a second graph representation based on the subset of the plurality of sequence reads associated with the individual and the first graph representation;
(e) identify one or more candidate variants based on the second graph representation;
(f) add the identified one or more candidate variants to the set of genomic variants;
(g) determine whether one or more of a plurality of termination conditions is met, in accordance with a determination that none of the plurality of termination conditions is met:
   increment the node length by a predefined value; and
   repeat steps (c)-(g);
in accordance with a determination one or more of the plurality of termination conditions are met:
   forego repeating steps (c)-(g).

Although the disclosure and examples have been fully described with reference to the accompanying figures, it is to be noted that various changes and modifications will become apparent to those skilled in the art. Such changes and modifications are to be understood. as being included within the scope of the disclosure and examples as defined by the claims.

The foregoing description, for purpose f explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

What is claimed is:

1. A computer-enabled method for identifying a set of genomic variants in an individual, the method comprising:
(a) receiving a plurality of sequence reads associated with the individual;
(b) identifying, based on a locus of interest on a reference sequence, a subset of the plurality of sequence reads associated with the individual;
(c) constructing a first graph representation of at least a portion of the reference sequence, wherein the first graph representation comprises a plurality of reference nodes and wherein each reference node of the plurality of reference nodes has a same node length;
(d) constructing a second graph representation based on the subset of the plurality of sequence reads associated with the individual and the first graph representation;
(e) identifying one or more candidate variants based on the second graph representation;
(f) adding the identified one or more candidate variants to the set of genomic variants;
(g) determining whether one or chore of a plurality of termination conditions is met, in accordance with a determination that none of the plurality of termination conditions is:
incrementing the node length y a predefined value; and
repeating steps (c)-(g);
in accordance with a determination one or more of the plurality of termination conditions are met:
foregoing repeating steps (c)-(g).

2. The method of claim 1, wherein each of the first and the second graph representations is a De Bruijn graph.

3. The method of any of claims 1-2, wherein the plurality of sequence reads associated with an individual are from a sample acquired from the individual.

4. The method of any of claims 1-3, wherein the individual has a cancer chosen from a bladder cancer, a brain cancer, a breast cancer, a colon cancer, a hemangioblastoma, a liver cancer, a lung cancer, a melanoma, a neuroendocrine cancer, a pancreatic cancer, a retinoblastoma, a stomach cancer, a thyroid cancer, a uterine or endometrial cancer, a Wilms' tumor, or an ovarian cancer.

5. The method of any of claims 1-4, further comprising: before step (e),
identifying a reference node having more than one instance in the first graph representation; and
marking the reference node as an ambiguous node.

6. The method of claim 5, further comprising: associating the reference node with a reconsideration list.

7. The method of any of claims 1-7, wherein identifying one or more candidate variants based on the second graph representation comprises: traversing a path diverging from a reference node in the second graph representation until a traversal termination condition of a plurality of traversal termination conditions is met.

8. The method of claim 8, wherein the traversal termination comprises a determination that the path includes an ambiguous node.

9. The method of claim 9, further comprising: foregoing adding a candidate variant to a plurality of candidate variants based on the path.

10. The method of claim 8, wherein the traversal termination comprises a determination that the path includes a cycle.

11. The method of claim 11, further comprising: foregoing adding a candidate variant to a plurality of candidate variants based on the path.

12. The method of any of claims 10-11 further comprising: associating the cycle with a reconsideration list.

13. The method of claim 8, wherein the traversal termination comprises a determination that the path includes a dead end.

14. The method of claim 13, further comprising: adding a candidate variant to a plurality of candidate variants based on the path.

15. The method of claim 8, wherein the traversal termination comprises a determination that the path joins a reference node that is not an ambiguous node.

16. The method of claim 15, further comprising: adding a candidate variant to a plurality of candidate variants based on the path.

17. The method of any of claims 1-16, wherein identifying one or more candidate variants based on the second graph representation comprises:
obtaining a plurality of candidate variants; and
clustering the plurality of candidate variants.

18. The method of claim 17, further comprising: updating the plurality of candidate variants by removing candidate variants belonging to a problematic cluster.

19. The method of claim 18, wherein the problematic cluster is identified based on one or more predefined rules.

20. The method of claim 18, further comprising: updating the plurality of candidate variants by decomposing one or more candidate variants in the plurality of candidate variants.

21. The method of any of claims 1-20, wherein the plurality of termination conditions comprises a determination that node length exceeds a threshold.

22. The method of any of claims 1-20, wherein the plurality of termination conditions comprises a determination that no nodes or edges are associated with a reconsideration list.

23. The method of any of claims 1-22, further comprising: classifying a genomic variant of the set of genomic variants to one of a plurality of categories.

24. The method of any of claims 23, wherein the plurality of categories comprises an insertion, a deletion, a substitution, a rearrangement, or any combination thereof.

25. The method of any of claims 1-23, further comprising: identifying a variant of interest from the set of genomic variants.

26. The method of claim 25, further comprising: directing a treatment based on the variant of interest.

27. The method of claim 25, further comprising: providing an output indicative of a diagnosis based on the variant of interest.

28. The method of any of claims 1-27, further comprising: providing one or more textual or graphical outputs based on the one or more candidate variants.

29. The method of any of claims 1-28, wherein identifying, based on the locus of interest on the reference sequence, the subset of the plurality of sequence reads associated with the individual comprises: conducting a preliminary alignment of the plurality of sequence reads with respect to the reference sequence.

30. An electronic device, comprising:
one or more processors;
a memory; and
one or more programs, wherein the one or more programs are stored in the memory and configured to be executed by the one or more processors, the one or more programs including instructions for:
(a) receiving a plurality of sequence reads associated with the individual;
(b) identifying, based on a locus of interest on a reference sequence, a subset of the plurality of sequence reads associated with the individual;
(c) constructing a first graph representation of at least a portion of the reference sequence, wherein the first graph representation comprises a plurality of reference nodes and wherein each reference node of the plurality of reference nodes has a same node length;
(d) constructing a second graph representation based on the subset of the plurality of sequence reads associated with the individual and the first graph representation;
(e) identifying one or more candidate variants based on the second graph representation;
(f) adding the identified one or more candidate variants to the set of genomic variants;
(g) determining whether one or more of a plurality of termination conditions is met, in accordance with a determination that none of the plurality of termination conditions is met:
incrementing the node length by a predefined value; and
repeating steps (c)-(g);
in accordance with a determination one or more of the plurality of termination conditions are met:
foregoing repeating steps(c)-(g).

31. A non-transitory computer-readable storage medium storing one or more programs, the one or more programs comprising instructions, which when executed by one or more processors of an electronic device having a display, cause the electronic device to:
(a) receive a plurality of sequence reads associated with the individual;
(b) identify, based on a locus of interest on a reference sequence, a subset of the plurality of sequence reads associated with the individual;
(c) construct a first graph representation of at least a portion of the reference sequence, wherein the first graph representation comprises a plurality of reference nodes and wherein each reference node of the plurality of reference nodes has a same node length;
(d) construct a second graph representation based on the subset of the plurality of sequence reads associated with the individual and the first graph representation;
(e) identify one or more candidate variants based on the second graph representation;
(f) add the identified one or more candidate variants to the set of genomic variants;
(g) determine whether one or more of a plurality of termination conditions is met, in accordance with a determination that none of e plurality of termination conditions is met:
increment the node length by a predefined value; and
repeat steps (c)-(g);
accordance with a determination one or more of the plurality of termination conditions met:
forego repeating steps (c)-(g).


SEQUENCE LISTING (4 sequences)

SEQ ID NO: 1 - DNA, 12 bp, Artificial Sequence
  Synthetic Construct
acgtaaacgtag

SEQ ID NO: 2 - DNA, 12 bp, Artificial Sequence
  Synthetic Construct
acgtaaacgtga

SEQ ID NO: 3 - DNA, 12 bp, Artificial Sequence
  Synthetic Construct
acgtaatcgtag

SEQ ID NO: 4 - DNA, 12 bp, Artificial Sequence
  Synthetic Construct
acgtaatcgtga